(19) United States
(12) Patent Application Publication
(10) Pub. No.: US 2002/0114786 A1
(43) Pub. Date: Aug. 22, 2002

(54) Pharmaceutical preparations comprising soybean isoflavone extracts and probiotic microogranisms

(76) Inventors: Pierre Fabre, Castres (FR); Rene Belle, Rivieres (FR); Bernard Fabre, Belberaud (FR)

Correspondence Address: THE FIRM OF HUESCHEN AND SAGE, 500 COLUMBIA PLAZA, 350 EAST MICHIGAN AVENUE, KALAMAZOO, MI, 49007, US

(21) Appl. No.: 09/951,690

(22) Filed: Sep. 14, 2001

(30) Foreign Application Priority Data
Sep. 11, 2000 (FR) 0011515

(51) Int. Cl. A61K 35/78

(52) U.S. Cl. 424/93.3; 424/757

(57) ABSTRACT

Pharmaceutical preparations comprising soybean extracts which are standardized for isoflavones and live probiotic microorganisms, as a combination product for administration which is simultaneous, separate or staggered in time, which can be used in treating the symptoms linked to the menopause.

The present invention relates to combinations of soybean isoflavones and probiotic microorganisms, and to their use in therapy.

Soybean belongs to the Fabaceae family. The soybean fruit is a pod containing round or ovoid seeds which are enclosed in twos, threes or fours. These soybean seeds are mainly composed of carbohydrates, proteins and lipids. The carbohydrates, which represent 15 to 25% of the substance, consist of simple sugars, such as sucrose, raffinose and stachyose, of pentosams and galactosanns but very little starch. Proteins are present in abundance since they represent 35 to 40% of the substance and principally consist of albumins and globulins. It is this wealth of protein which makes soybean seeds nutritionally valuable. Lipids correspond to 15 to 20% of the substance and principally consist of saturated or unsaturated fatty acid glycerides, of a phytosterol-rich unsaponifiable fraction and of a phospholipid-rich polar lipid fraction termed lecithin.

While isoflavones are also an important constituent of soybean seeds, the content of isoflavones in soybean varies considerably depending on the species, the maturity of the plant and the culture conditions. These isoflavones are heterocyclic polyphenols which are characterized by a C15 chain of the Ar-C₃-Ar type arranged in accordance with a 1,2-diphenylpropane motif. The general structure is given in formula (1) below:

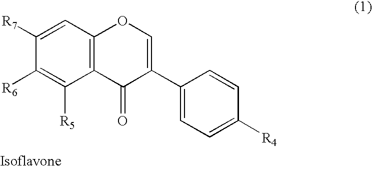

In the plant, the main isoflavones, i.e. genistein of the formula (2)

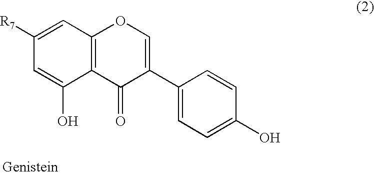

and daidzein of the formula (3), where R₇=hydrogen,

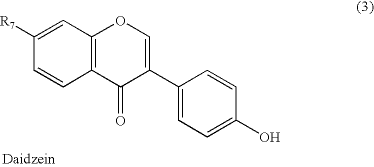

are present in different forms: glycosides, malonides, acetylated forms and aglycones, with this latter form being in a minority since it only represents 1 to 2% of the total isoflavones. The conjugations take place at position 7.

Wang H. et al. (J. Agric. Food Chem. (1994) 42, No. 8, pages 1666 to 1673) found that 2.275 mg of isoflavones, in all the abovementioned forms, were present per gram of soybean, corresponding to daidzein and genistein contents of 43% and 57%, respectively.

Tham D. M. et al. (J. Clin. Endocrinol. Metab. (1998) 83, No. 7, pages 2232 to 2235) assayed two different species and found concentrations of 1.005 mg/g and 4.031 mg/g, with daidzein contents of 36% and 32%, respectively.

In fact, a value of 2 mg of isoflavones per gram of dry soybean seed is generally accepted.

The structure of the isoflavones exhibits homologies with the structures of estrogens. Thus, without being steroids, genistein and daidzein possess hydroxyl groups in positions 7 and 4′ in a stereochemical configuration which is analogous to that of the hydroxyls in the estradiol molecule of the formula (4).

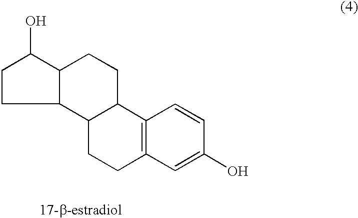

Due to this structural homology with estradiol, and more specifically due to the distance and the dihedral angle between the two hydroxyl groups, the isoflavones possess estrogenic activity. Thus, while they are able to bind, like estradiol, to type II (β) receptors (Bennetau-Pelissero Lettre Scientifique de l'Institut Francais pour la Nutrition [Scientific Letter of the French Institute for Nutrition] (1997) 47), this activity is at least 500 times weaker than that of estradiol.

Thus, if the activity of estradiol is set at 100, daidzein then has an activity of 0.013, genistein has an activity of 0.084 and equol, which is a metabolic derivative of daidzein, has an activity of 0.61 (Markiewicz L., J. Steroid Biochem. Molec. Biol. (1993) 45, pages 399 to 405).

Soybean is eaten by Asiatic populations, more specifically in fermented forms such as miso or tempeh.

Observation of these populations, which therefore have a diet which is very rich in phytoestrogens and consume between 20 and 150 mg of isoflavones per day Murkies A. et al. (J. Clin. Endocrinol. Metab. (1998) 83, No. 2, pages 297 to 303), has shown differences with regard to health when compared with Western populations. These Asiatic populations display:

4 to 10 times less probability of dying of a hormone-dependent cancer (breast, endometrium, prostate and colon),

fewer symptoms correlated with hormonal disorders at the time of menopause (osteoporosis, hot flushes), and

fewer coronary and heart diseases.

On account of their structural homology with estrogens, isoflavones are often used in medicine, principally in medical gynecology. Thus, Wilcox C. et al. (BMJ (1990) 24, pages 1757 to 1761) and Washburn S. A. et al. (Third International Conference on Phytoestrogens (1995) Little Rock, USA) have respectively demonstrated, in controlled studies carried out against a placebo, that hot flushes decrease substantially and significantly when soybean is ingested at the rate of 40 g per day for 6 weeks. Albertazzi P. et al. (Am. J. Obstet. Gynecol. (1998) 91, pages 6 to 11) have confirmed this effect with 60 g of soybean (that is 76 mg of isoflavones) per day in a randomized study carried out on more than 100 menopausal women. These studies consequently demonstrate the effect of phytoestrogens on hot flushes.

Furthermore, Valente M. et al. (Calcif. Tissue Int. (1994) 54, pages 377 to 380) have carried out studies in women using ipriflavone, a synthetic prodrug of daidzein, which was given at the rate of 500 to 600 mg per day, with these studies showing bone gain at the level of L1-L5, with a return to normal in 12 months, in 40 menopausal women suffering from osteoporosis. Gambacciani M. et al. (Bone and mineral, 26, pages 19 to 26) have shown that it is also possible to avoid bone losses in women being treated with LHRH (luteinizing hormone releasing hormone) agonists. Blair et al. (J. Cell. Biochem., 1996, 61, pages 629 to 637) explain this action by demonstrating that genistein inhibits osteoclastic activity.

In the case of hormonal dependent cancers, Barnes S. et al., (Diet and cancer: markers, prevention and treatment (1994) Plenum Press, pages 135 to 147) have shown a reduction in the number and size of tumors. Fundamental studies carried out by Pagliacci N. C. et al. (European J. of Cancer (1994) 30A (11) pages 1675 to 1682) have demonstrated that phytoestrogens inhibit human mammary cancer cells while Wutke W. (in Proceedings of the 2nd Congress of the European Society of Gynecology (1997)) have demonstrated inhibition of the expression of genes responsible for the growth of cell cultures. Finally, the study carried out by Lee H. P. et al. (Lancet (1991) 337, No. 18, pages 1197 to 1200) has shown that the risk of breast cancer falls when menopausal women consume a soybean-rich diet.

The small intestine is the preferential site for the absorption of isoflavones because the contact surface is large and the vascular network is dense. However, only the aglycone forms (genistein and daidzein) are absorbed because the hydrophilicity and molecular weight of the glycosides are too great.

It has been demonstrated that it is only the aglycone forms which have estrogenic activity in animals (Tham D. M. et al., loc. cit., and Moreau L. et al. (References en gynécologie obstetrique [References in obstetric gynecology] (1999), pages 229 to 234, Springer-Verlag). The biotransformation of the glycosylated isoflavones into equol, genistein and daidzein is therefore necessary in order to observe the beneficial effects of soybean.

Chang Y. C. et al. (J. Nat. Prod. (1995) 58, No. 12, pages 1892 to 1896) have shown that the intestinal microflora performs this necessary transformation of the glycosylated isoflavones (daidzine and genistine) into daidzein, genistein and equol.

In order to make use of the isoflavones, the intestinal flora therefore contains bacteria which are able to hydrolyze the β1-4 bonds and thereby release the aglycone forms. While these microorganisms secrete enzymes such as β-glucosidases, this deglycosylation is also partially effected by simple hydrolysis by the hydrochloric acid of the stomach.

Finally, the molecules which have already been absorbed, and are therefore conjugated, can be deconjugated and reabsorbed using the enzymes β-glucuronidase and arylsulfurase.

The diversity of the intestinal microflora, in particular the Lactobacilli, the Bacteroides and the Bifidobacteria, plays an important role in the pathways of metabolism and the bioavailability of the isoflavones (Xu X. et al. (1995) Bioavailability of soybean isoflavons depends upon gut microflora in women I, Nutro 125, pages 2307 to 2315).

In general, the conversion is efficient and only 14 to 43% of the ingested isoflavones are recovered intact in the urine.

However, there is great individual metabolic diversity, and Moreau et al. (References en gynécologie obstetrique, loc. cit.) have shown that 30% of the population does not possess the flora which is able to metabolize equol, i.e. the most effective aglycone form as regards the estrogenic effects, thereby making treatment with isoflavones difficult.

Probiotic organisms are “living organisms which, when ingested in sufficient quantity, exert beneficial effects on health which go beyond the inherent nutritional benefits”. The benefits to health are not limited to improvement of the microbial balance at the level of the intestine but can also include other effects. Thus, in the case of the lactic acid bacteria, five types of effect are observed:

Effects on the digestive tract: improvement in the digestion of lactose in individuals who are deficient in lactase, prevention of intestinal disturbances, adherence to cultures of intestinal cells, stimulation of intestinal immunity in animal models, stabilization of Crohn's disease and regulation of intestinal motility.

Alteration of the microflora: equilibration of intestinal bacteria, increase in fecal bacteria, decrease in the activity of fecal enzymes, decrease of fecal mutagenicity, relief of symptoms of intolerance to lactose and colonization of the digestive tract.

Effect on diarrhea: prevention and/or treatment of acute diarrheas and diarrheas due to rotavirus, decrease in diarrheas due to rotavirus, prevention of diarrhea associated with antibiotics, treatment of recurrent diarrheas due to Clostridium difficile and treatment of persistent diarrheas in children.

Systemic effects: stimulation of phagocytic activity, stimulation of interferon γ production by cultures of human blood monoculear cells, reduction of hypertension in animal and human models, beneficial effects on superficial cancer of the bladder and release of clinical symptoms in children suffering from optical dermatitis.

Physiological effects: production of bacteriocins and antagonist effect on Helicobacter pylori.

As far they are concerned, the Bifidobacteria improve gastrointestinal transit, decrease the risk of cancer of the colon, and possess antimicrobial activity and immunomodulating effects, while Lactobacillus acidophilus possesses hypocholesterolemic, anti-microbial and immunomodulating effects, while at the same time preventing cancer of the colon.

Consequently, it appears necessary to make up for the weak estrogenic activity of the isoflavones and to improve their activity in female patients who are exhibiting premenopausal and/or menopausal symptoms and, more specifically, in female patients who are not particularly responsive or who are not responsive at all.

The inventors have now found, surprisingly, that, when combined with isoflavones, certain probiotic microorganisms are able to solve this problem.

The present invention consequently relates to pharmaceutical preparations which comprise soy bean extracts which are standardized for isoflavones and live probiotic microorganisms as a combination product for administration which is simultaneous, separate or staggered in time and which can be used in the treatment of symptoms linked to the menopause.

In one particular embodiment, the isoflavone extract is standardized to an isoflavone content of from 10 to 50%.

In another particular embodiment, the probiotic microorganisms are selected from the group consisting of Enterococcus faecium, Propionibacterium acidipropionici, Lactobacillus acidophilus, Lactobacillus brevis, Lactobacillus plantarum, Lactobacillus casei, Lactobacillus rhamnosus, Pediococcus acidilactici and Streptococcus thermophilus, and mixtures of these microorganisms.

In one particularly advantageous embodiment of the invention, the preparations comprise the isoflavone extract which is standardized to an isoflavone concentration of between 10 and 50% and the probiotic microorganisms are selected from the group consisting of Enterococcus faecium, Propionibacterium acidipropionici, Lactobacillus acidophilus and Lactobacillus brevis.

Within the meaning of the present invention, a soybean extract standardized for isoflavones is understood as meaning an extract which is prepared as follows: the ground soybean seeds are extracted with a water-alcohol solution. After the alcohol has been removed, the aqueous solution is defatted using an organic solvent and then purified on a column containing an absorbent resin.

The probiotic microorganisms are selected on the basis of their biological properties and on their abilities to metabolize isoflavones.

These microorganisms can be used in the form of commercially available cultures, in the form of a microbial suspension or, preferably, in the form of a lyophilizate which is prepared under standard conditions known to a skilled person.

In one particularly advantageous embodiment of the invention, these microorganisms are present at the rate of from 50.10⁹ to 1.10⁶ bacteria per unit dose, and the isoflavone extract is present at the rate of from 100 to 300 mg per unit taken.

The preparations in accordance with the invention can be used, in particular, as a drug, particularly for treating symptoms linked to the menopause, particularly against hot flushes.

The preparations according to the invention comprising the soybean extract and the microorganisms can be presented in any customarily employed dry or liquid form, in particular in the form of lozenges, tablets, capsules, gelatin capsules or potable solutions in combination with any pharmaceutically acceptable excipient.

The isoflavone extract and the probiotic microorganisms can also be administered in the form of separate unit doses, in particular lozenges, tablets, capsules, gelatin capsules or potable solutions which can be administered separately or simultaneously or else staggered in time.

The preparations according to the invention enable the glycosylated forms to be metabolized into active aglycones from the time they are first absorbed, which is in the duodenum, which is the region of maximum absorption in the digestive tract.

The examples which follow illustrate the invention without, however, limiting it.

### EXAMPLE 1

### Formulation Examples

Formulation 1:

soybean extract standardized to an isoflavone content of 15%, 250 mg in a size 1 gelatin capsule,

lyophilizate of Lactobacillus acidophilus, 10⁹ bacteria in a size 1 gelatin capsule

take 1 gelatin capsule of each simultaneously per day.

Formulation 2:

soybean extract standardized to an isoflavone content of 15%, 100 mg,

lyophilizate of Lactobacillus acidophilus, 10⁶ bacteria in one and the same size 1 gelatin capsule

take 2 gelatin capsules per day, i.e. 1 in the morning and 1 in the evening, or 2 in the morning or 2 in the evening.

Formulation 3:

soybean extract standardized with an isoflavone content of 40%, 200 mg, formulated in a tablet,

lyophilizate of Lactobacillus brevis, 10⁹ bacteria in a size 1 gelatin capsule

take 1 gelatin capsule of each simultaneously per day.

Formulation 4:

soybean extract standardized to an isoflavone content of 10%, 200 mg in a capsule,

lyophilizate of Enterococcus faecium, 10⁹ bacteria in a capsule

take 4 capsules of each simultaneously per day.

Formulation 5:

2% water-alcohol solution of soybean extract standardized to an isoflavone content of 50%,

microbial suspension of Lactobacillus acidophilus containing 10⁸ organisms/g,

using a calibrated spoon, take 10 ml of each of the solutions per day.

### EXAMPLE 2

### Selecting Isoflavone-metabolizing Microorganisms

1) Method of operation:

Microorganisms which are present in the fecal flora, and which are able to metabolize genistine and daidzine into genistein and daidzein, respectively, were cultured for 24, 48 and 72 hours in a nutrient medium containing: meat peptone 10 g/l, meat extract 10 g/l, yeast extract: 5 g/l, glucose 20 g/l, sodium acetate 5 g/l, Tween 80 l g/l, dipotassium phosphate 2 g/l, ammonium citrate 2 g/l, magnesium sulfate 0.2 g/l, manganese sulfate 0.05 g/l, and a soybean extract. At each of these times, a sample is removed from the microbial cultures, diluted with an equal volume of methanol and then filtered. In each case, the methanolic solution is analyzed by high performance liquid chromatography. High performance liquid chromatography is used to assay daidzine, genistine, daidzein and genistein in the initial extract and in the media metabolized by the different microorganisms. In this way, the contents of the aglycone and glycosylated forms in the metabolized extract are calculated with respect to the glycosylated forms (100%) in the initial extract.

2) Results

These are compiled in Table 1 below.

TABLE 1

| | | Propioni- | Lacto- | | | Pedio- | Strepto- | | |
|---|---|---|---|---|---|---|---|---|---|
| | Entero- | bacterium | bacillus | Lacto- | Lacto- | coccus | coccus | Lacto- | Lacto- |
| | coccus | acidipro- | acido- | bacillus | bacillus | acidi- | thermo- | bacillus | bacillus |
| | faecium | pionici | philus | brevis | plantarum | lactici | philus | casei | rhamnosus |
| Genistine | 5.5% | 12% | 6.5% | 2% | 53% | 63% | 73% | 85% | 5% |
| Daidzine | 0.1% | 33% | 16% | 16% | 75% | 77% | 65% | 80% | 26% |
| Genistein | 80% | 72% | 83% | 81% | 44% | 30% | 25% | 13% | 85% |
| Daidzein | 85% | 57% | 84% | 82% | 22% | 23% | 33% | 18% | 71% |

These microorganism strains metabolize approximately 80% of the glycoconjugated forms of the isoflavones into aglycone forms which are easily absorbed in the intestine; Enterococcus faecium, Propionibacterium acidipropionici, Lactobacillus acidophilus and Lactobacillus brevi turn out to be the most active strains.

### EXAMPLE 3

### Clinical Assessment

1) Way in which study was carried out:

The study was performed with the aim of testing the efficacy of the soybean-probiotic organism combination, using the hot flush, in the indication of menopause, as the criterion of efficacy and employing 2 groups of menopausal women who were given a soybean extract which was or was not combined with probiotic organisms.

This study lasted for 3 months, together with a preinclusion period of one month.

One group of 50 menopausal women were treated, once per day, with 1 gelatin capsule containing 10⁹ Lactobacillus acidophilus bacteria/g. Another group of 50 menopausal women were given, once per day, 1 gelatin capsule containing 250 mg of soybean extract having an isoflavone content of 15% and one placebo gelatin capsule.

Each subject noted, in a self-assessment diary, the daily number of hot flushes and their intensity in accordance with a preestablished scale.

2) Results:

This study demonstrates the positive and important role played by probiotic organisms in determining the regularity and intensity of the response to phytoestrogens, since the preparations according to the invention enable the women who were given the soybean extract combined with the probiotic organisms to observe a decrease in the number and intensity of hot flushes which was greater than that observed by the women who were given the soybean extract alone.

1. A pharmaceutical preparation which comprises a soybean extract which is standardized for isoflavones and live probiotic microorganisms, as a combination product for administration which is simultaneous, separate or staggered in time, which can be used in treating the symptoms which are linked to the menopause.

2. The preparation as claimed in claim 1, wherein the isoflavone extract is standardized to an isoflavone content of between 10 and 50%.

3. The preparation as claimed in claim 1 or 2, wherein the probiotic microorganisms are selected from the group consisting of Enterococcus faecium, Propionibacterium acidipropionici, Lactobacillus acidophilus, Lactobacillus brevis, Lactobacillus plantarum, Lactobacillus casei, Lactobacillus rhamnosus, Pediococcus acidilactici and Streptococcus thermophilus, and mixtures of these microorganisms.

4. The preparation as claimed in claims 2 and 3, wherein the isoflavone extract is standardized to an isoflavone content of between 10 and 50% and the probiotic microorganism are selected from the group consisting of Enterococcus faecium, Propionibacterium acidpropionici, Lactobacillus acidophilus and Lactobacillus brevis.

5. The preparation as claimed in any one of claims 1 to 4, wherein the probotic microorganisms are used in the form of a microbial suspension or of a lyophilizate.

6. The preparation as claimed in any one of claims 1 to 5, wherein the probiotic microorganisms are present at a concentration of between 50.10⁹ and 1.10⁶ bacteria per unit taken.

7. The preparation as claimed in any one of claims 1 to 6, wherein the isoflavone extract is present at the rate of 100 to 300 mg per unit taken.

8. The use of a preparation as claimed in any one of claims 1 to 7 for preparing a drug which can be used for treating the symptoms linked to the menopause.

9. The use as claimed in claim 8, wherein the symptoms are hot flushes.
